(19) United States
(12) Patent Application Publication
(10) Pub. No.: US 2021/0057043 A1
(43) Pub. Date: Feb. 25, 2021

(54) TUMOR NEOANTIGEN PREDICTION PLATFORM AND APPLICATION THEREOF IN NEOANTIGEN VACCINE DEVELOPMENT SYSTEM

(71) Applicant: Shenzhen Neocura Biotechnology Corporation, Shenzhen (CN)

(72) Inventors: Youdong PAN, Shenzhen (CN); Qi SONG, Shenzhen (CN); Ji WAN, Shenzhen (CN); Jun-Yuan HUANG, Shenzhen (CN); An XIAO, Shenzhen (CN); Gang LIU, Shenzhen (CN); Ying WEN, Shenzhen (CN)

(73) Assignee: Shenzhen Neocura Biotechnology Corporation, Shenzhen (CN)

(21) Appl. No.: 16/904,582

(22) Filed: Jun. 18, 2020

(30) Foreign Application Priority Data
Aug. 23, 2019 (CN) 201910785281.9

(51) Int. Cl. G16B 25/20; C07K 16/28; G16B 30/10; C12N 15/86; G16B 50/00; H01J 49/26

(52) U.S. Cl. CPC G16B 25/20; C07K 16/2833; G16B 30/10; C12N 2740/10043; G16B 50/00; H01J 49/26; C12N 15/86

(57) ABSTRACT

A tumor neoantigen prediction platform and an application thereof in a neoantigen vaccine development system are provided. The present invention selects 55 HLA-A and HLA-B subtypes with a high proportion of Chinese population from a common database, then establishes a method for constructing cell lines expressing associated HLA subtypes, and subsequently analyzes the resulting HLA subtype cell line binding proteomes by protein mass spectrometry at an attomolar (10⁻¹⁸ molar) level; through very high-precision mass spectrometry of protein profiling presented on the surface of a single cell, the present invention builds a high-frequency HLA-binding polypeptide database for Chinese population; subsequently, a tumor neoantigen prediction algorithm is optimized by a prediction platform including the HLA-binding polypeptide database, thereby significantly improving the tumor neoantigen prediction accuracy. The prediction platform in the tumor neoantigen vaccine development system can be used to help improve the efficiency of selection, research and development of vaccines.

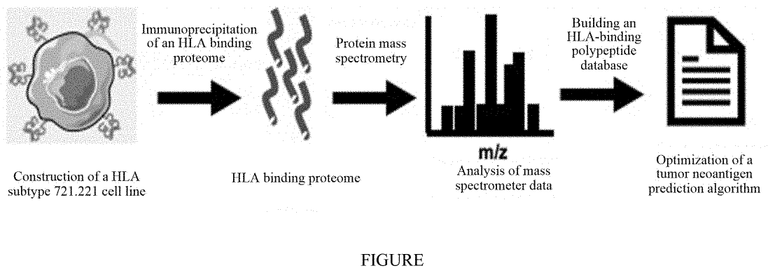

### CROSS REFERENCE TO THE RELATED APPLICATIONS

This application is based upon and claims priority to Chinese Patent Application No. 201910785281.9, filed on Aug. 23, 2019, the entire contents of which are herein incorporated by reference.

### TECHNICAL FIELD

The present invention relates to the fields of molecular biology and bioinformatics, and in particular to a tumor neoantigen prediction platform and an application thereof in a neoantigen vaccine development system. Specifically, the tumor neoantigen prediction platform of the present invention includes a human leukocyte antigen (HLA)-binding polypeptide database. The HLA-binding polypeptide database is built after analyzing 55 HLA binding proteomes with a high proportion of Chinese population by high sensitivity protein mass spectrometry. The prediction platform of the present invention significantly improves the tumor neoantigen prediction accuracy by optimizing a tumor neoantigen prediction algorithm. The prediction platform can also be used in a tumor neoantigen vaccine development system to improve the efficiency of selection, research and development of vaccines.

### BACKGROUND

Tumor neoantigen refers to a specific polypeptide generated by gene mutation of a tumor cell, which is presented to the cell surface in an immune fashion by HLA and thus recognized by a T lymphocyte to elicit an immune response. Neoantigens are extremely suitable to serve as markers for immunotherapy and further used to develop tumor neoantigen vaccines, because these neoantigens are generated by tumor cell mutation but not expressed in normal cells.

However, the development and application of tumor neoantigen vaccines still face various challenges so far. Generally, the common prediction algorithms (e.g., NetMHCpan) have excessively low accuracy, thereby influencing the efficiency of the selection, research and development of vaccines. The above disadvantage greatly limits the promotion and application of the tumor neoantigen and vaccines thereof in the field of oncology.

### SUMMARY

To overcome the disadvantage of low accuracy in common prediction algorithms and improve the efficiency of selection, research and development of vaccines, the present invention selects 55 HLA-A and HLA-B subtypes with a high proportion of Chinese population from a common database, then establishes a method for constructing cell lines expressing associated HLA subtypes, and subsequently analyzes the resulting HLA subtype cell line binding proteome by protein mass spectrometry at an attomolar (10⁻¹⁸ molar) level. Through extremely high-precision mass spectrometry of protein profiling presented on the surface of a single cell, the present invention builds a high-frequency HLA-binding polypeptide database for Chinese population. Subsequently, a tumor neoantigen prediction algorithm is optimized by a prediction platform including the HLA-binding polypeptide database, thereby significantly improving the tumor neoantigen prediction accuracy. The platform in the tumor neoantigen vaccine development system can be used to help improve the efficiency of the selection, research and development of vaccines.

To achieve the above objective, the present invention provides a tumor neoantigen prediction platform, where the prediction platform includes an HLA-binding polypeptide database.

Further, the HLA-binding polypeptide database is built by analyzing the binding proteomes of MHC-negative 721.221 cell lines expressing each of 55 HLA subtypes with a high proportion of Chinese population by high sensitivity protein mass spectrometry at an attomolar level, and the 55 HLA subtypes include 24 HLA-A subtypes and 31 HLA-B subtypes, specifically numbered as follows: HLA-A*01:01, HLA-A*02:01, HLA-A*02:02, HLA-A*02:03, HLA-A*02:04, HLA-A*02:05, HLA-A*02:06, HLA-A*02:07, HLA-A*03:01, HLA-A*11:01, HLA-A*11:02, HLA-A*23:01, HLA-A*24:02, HLA-A*24:03, HLA-A*26:01, HLA-A*29:02, HLA-A*30:01, HLA-A*31:01, HLA-A*32:01, HLA-A*33:01, HLA-A*33:03, HLA-A*66:01, HLA-A*68:01, HLA-A*68:02, HLA-B*07:02, HLA-B*08:01, HLA-B*13:01, HLA-B*13:02, HLA-B*15:01, HLA-B*15:02, HLA-B*15:10, HLA-B*27:02, HLA-B*27:03, HLA-B*27:05, HLA-B*27:06, HLA-B*35:01, HLA-B*38:01, HLA-B*38:02, HLA-B*39:01, HLA-B*39:09, HLA-B*39:011, HLA-B*40:01, HLA-B*40:02, HLA-B*40:06, HLA-B*44:02, HLA-B*44:03, HLA-B*46:01, HLA-B*48:01, HLA-B*51:01, HLA-B*52:01, HLA-B*53:01, HLA-B*54:01, HLA-B*55:02, HLA-B*57:01, and HLA-B*58:01.

Further, a method for building the HLA-binding polypeptide database includes the following steps:

(1) performing an immunoprecipitation on the 721.221 cell lines expressing the 55 HLA subtypes with anti-human HLA monoclonal antibodies, and isolating HLA binding proteomes, followed by an elution and a desalting;

(2) analyzing the isolated HLA binding proteomes by high sensitivity protein mass spectrometry at an attomolar level; and

(3) building the HLA-binding polypeptide database by mass spectrometry data of the HLA-binding proteomes.

Further, each of the MHC-negative 721.221 cell lines expressing one of the HLA subtypes for building the prediction platform of the present invention is prepared by the following steps:

(1) replicating an HLA gene fragment from an HLA homozygous human B-lymphocyte cell line with HLA locus-specific primers, and validating the correctness of a sequence of the PCR fragment;

(2) cloning the HLA PCR fragment into a retrovirus vector pLNCX2 and preparing a retrovirus; and

(3) infecting a 721.221 cell line with a cell culture medium containing the retrovirus, and picking out a resulted 721.221 cell line stably expressing an HLA subtype by a FACSAria cell sorter.

Further, the prediction platform of the present invention improves the tumor neoantigen prediction accuracy by optimizing a tumor neoantigen prediction algorithm, thus improving the efficiency of the selection, research and development of vaccines.

The present invention further relates to an application of the prediction platform in a neoantigen vaccine development system.

The present invention further relates to an application of the prediction platform in oncology.

In another aspect, the present invention further provides a method for constructing an MHC-negative 721.221 cell line expressing one of the HLA subtypes, including the following steps:

(1) replicating an HLA gene fragment from an HLA homozygous human B-lymphocyte cell line with HLA locus-specific primers, and validating the correctness of a sequence of the PCR fragment;

(2) cloning the HLA PCR fragment into a retrovirus vector pLNCX2 and preparing a retrovirus; and

(3) infecting a 721.221 cell line with a cell culture medium containing the retrovirus, and picking out a resulted 721.221 cell line stably expressing the HLA subtype by a FACSAria cell sorter.

The present invention further relates to an application of the method in biomedicine.

Compared with the existing tumor neoantigen prediction techniques, the prediction platform of the present invention can optimize the tumor neoantigen prediction algorithm and thus significantly improve the tumor neoantigen prediction accuracy. The prediction platform can also be used in a tumor neoantigen vaccine development system to improve the efficiency of the selection, research and development of vaccines.

### BRIEF DESCRIPTION OF THE DRAWINGS

FIGURE shows a flowchart of building a high-frequency HLA-binding polypeptide database by a tumor neoantigen prediction platform of the present invention.

### DETAILED DESCRIPTION OF THE EMBODIMENTS

The following describes the present invention in detail through specific embodiments, but it should be noted that the following embodiments are merely exemplary. The present invention can also be implemented or applied through other different embodiments. Based on different viewpoints and applications, various modifications or amendments can be made to the embodiments without departing from the spirit of the present invention.

To enable those skilled in the art to understand the features and effects of the present invention, the following generally describes and defines the terms and dictions mentioned in the specification and claims. Unless otherwise specified, all technical and scientific terms used herein have the same meaning as commonly understood by a person of ordinary skill in the art to which the present invention pertains. In addition to the specific methods, devices, and materials used herein, any method, device, and material equivalent or similar to those described in the embodiments of the present invention can be used to implement the present invention by those skilled in the art according to conventional knowledge and the description of the present invention.

The materials, reagents, etc. used in the following embodiments are all commercially available, unless otherwise specified.

Embodiments

| Reagent used in the experiment | Supplier |
|---|---|
| HLA homozygous human | International |
| B-lymphocyte cell line | Histocompatibility Working |
| (B-lymphoblastoid cell line, B-LCL) | Group (IHWG) |
| 721.221 cell line | ATCC |
| Amphopack-293 cell line | ATCC |
| pLNCX2 | Takara |
| Lipofectamine | Thermo Fisher Scientific |
| G418 | Thermo Fisher Scientific |
| DNA polymerase | NEB |
| Anti-human HLA monoclonal | Biolegend |
| antibody (W6/32) | |
| HLA locus-specific primer | IDT |
| TA Cloning Kit | Thermo Fisher Scientific |
| Restriction enzymes HindIII, | Thermo Fisher Scientific |
| NotI, and EcoRI | |
| DNA sequencing primer | IDT |
| RPMI 1640 | Sigma-Aldrich |
| Fetal calf serum (FCS) | Thermo Fisher Scientific |
| Penicillin/Streptomycin with | Thermo Fisher Scientific |
| glutamine | |
| Sodium pyruvate | Thermo Fisher Scientific |
| RNeasy Mini Kit | Qiagen |
| Oligo dT Primer | Thermo Fisher Scientific |
| dNTP Mix | Thermo Fisher Scientific |
| DL-Dithiothreitol (DTT) | Thermo Fisher Scientific |
| MMLV reverse transcriptase | Thermo Fisher Scientific |
| Taq polymerase | Thermo Fisher Scientific |
| Gel Extraction Kit | Qiagen |
| INVaF′ competent cells | Thermo Fisher Scientific |
| LB/Ampicillin (100 μg/ml) | Sigma-Aldrich |
| DNA Miniprep Kit | Qiagen |
| Goat Anti-Mouse IgG FITC | Biolegend |
| DMEM | Sigma-Aldrich |
| GammaBind Plus Sepharose beads | GE Lifesciences |
| Empore C18 StageTips | 3M |
| Device | Supplier |
| Thermal cycler | Eppendorf |
| Protein mass spectrometer | Thermo Fisher Scientific |
| FACSAria Cell Sorter | BD |
| LSRII Flow Cytometer | BD |
| Sonicator | QSonica |

### Embodiment 1: Construction of an HLA Subtype 721.221 Cell Line

1. HLA locus-specific primers were used to replicate an HLA gene fragment from an HLA homozygous human B-lymphocyte cell line, including:

(1) Total RNA was extracted from the HLA homozygous human B-lymphocyte cell line (cultured in RPMI medium, supplemented with 10% FCS, 1% penicillin/streptomycin with glutamine, and 1 mM sodium pyruvate) by RNeasy Mini Kit.

(2) Oligo dT primer, dNTP Mix, DTT, MMLV reverse transcriptase were added to the extracted total RNA to prepare cDNA.

(3) 2 μl of the cDNA was mixed with PCR buffer, 200 μM of dNTP Mix, 0.3 μM of HLA focus-specific primer, and 0.5 d of Taq polymerase to conduct PCR.

The running program of the thermal cycler is listed in Table 1:

TABLE 1
The running program of the thermal cycler

| Step | Temperature | Time | Cycle |
|---|---|---|---|
| 1 | 95° C. | 5 min | 1 |
| 2 | 94° C. | 1 min | 30 |
| 3 | 58° C. | 1 min | |
| 4 | 68° C. | 1.5 min | |
| 5 | 68° C. | 4 min | 1 |

Sequences of HLA locus-specific primers are listed in Table 2:

TABLE 2
Sequences of HLA locus-specific primers

| SEQ | | | |
|---|---|---|---|
| ID | | | |
| NO: | Primer | Locus | Sequence (5′ to 3′) |
| 1 | A-5-HindIII | HLA-A | TATAAAGCTTGATTCT |
| | | | CCCCAGACGCCGAGG |
| 2 | B-5-HindIII | HLA-B | TATAAAGCTTCACCCG |
| | | | GACTCAGAGTCTCCT |
| 3 | A-3-NotI | HLA-A | ATATGCGGCCGCACAA |
| | | | GGCAGCTGTCTCACA |
| 4 | B-3-NotI | HLA-B | ATATGCGGCCGCATCT |
| | | | CAGTCCCTCACAAGA |

(4) PCR products were analyzed by 1.2% agarose gel electrophoresis, and then the PCR fragments were purified by Gel Extraction Kit.

(5) 6 μl of the purified PCR fragments were ligated to a pCR2.1 vector by TA Cloning Kit, the resulted vector was transformed into INVaF′ competent cells, and subsequently, the transformed INVaF′ competent cells were cultured on an LB/ampicillin plate, and placed at 37° C. overnight.

(6) A plurality of colonies was picked out from the LB/ampicillin plate, DNAs were extracted by DNA Miniprep Kit, and restriction enzyme EcoRI was used to check if the PCR fragments were inserted.

(7) M13 reverse and T7 promoter primers were further used for DNA sequencing to validate the correctness of sequences of the PCR fragments.

Sequences of DNA sequencing primers of the pCR2.1 vector are listed in Table 3:

TABLE 3
Sequences of DNA sequencing primers
of the pCR2.1 vector

| SEQ | | |
|---|---|---|
| ID | | |
| NO: | Primer | Sequence (5′ to 3′) |
| 5 | M13 reverse | CAGGAAACAGCTATGAC |
| 6 | T7 promoter | TAATACGACTCACTATAGGS |

2. The HLA PCR fragment was cloned into a retrovius vector pLNCX2 and preparing a retrovirus, including:

(1) The pCR2.1 vector with correct HLA sequence was cleaved with restriction enzymes HindIII and NotI, and the resulted HLA fragment was purified and cloned into the retrovirus vector pLNCX2.

(2) The resulted vector was sequenced with DNA sequencing primers of the pLNCX2 vector to validate the correctness of the sequence of the HLA fragment.

DNA sequencing primers of the pLNCX2 vector are listed in Table 4:

TABLE 4
DNA sequencing primers of the pLNCX2 vector

| SEQ | | |
|---|---|---|
| ID | | |
| NO: | Primer | Sequence (5′ to 3′) |
| 7 | 5-end | AGCTCGTTTAGTGAACCGTCAGATC |
| 8 | 3-end | ACCTACAGGTGGGGTCTTTCATTCCC |

(3) The pLNCX2 vector with the correct HLA sequence was transfected into an Amphopack-293 cell line to produce the retrovirus.

(4) Cell culture medium containing the retrovirus was collected 48 h after the transfection, and centrifuged at 2,000 rpm for 10 min; then, the supernatant was collected, filtered with a 0.45 μm filter membrane, and stored at 4° C. or frozen at −80° C. for subsequent use.

3. The 721.221 cell line was infected with the cell culture medium containing the retrovirus, and the resulted 721.221 cells stably expressing the HLA subtype were picked out on a FACSAria Cell Sorter, including:

(1) Lipofectamine was used to help the retrovirus infect the 721.221 cell line; one day after the infection, G418 was added to the cell culture medium (the final concentration of G418 was 800 μg/ml), followed by screening for 5-7 days.

(2) After the infection for 5-7 days is completed, a part of the resulted cells were incubated with an anti-human HLA monoclonal antibody (W6/32, 1:500) for 20 min at 4° C., and subsequently washed once with phosphate buffer.

(3) A part of the resulted cells were subjected to a second incubation with Goat Anti-Mouse IgG FITC (1:200) for 20 min at 4° C. and washed twice with phosphate buffer, and the HLA phenotype expressed on the cell surface was analyzed by LSRII Flow Cytometer.

(4) The 721.221 cells with the HLA phenotype expressed on the cell surface were cultured sequentially to obtain more cells; similarly, using the foregoing method, the cells were subjected to HLA staining, and the 721.221 cells with the HLA phenotype were selected by FACSAria Cell Sorter.

(5) The selected cells were cultured sequentially in a DMEM complete medium supplemented with 400 μg/ml of G418, and cryopreserved in a liquid nitrogen container.

### Embodiment 2: Analyzing 55 HLA Subtype MHC-Negative 721.221 Cell Line Binding Proteomes with a High Proportion of Chinese Population by High Sensitivity Protein Mass Spectrometry and Building an HLA-Binding Polypeptide Database

In the present invention, HLA subtype gene frequency database (http://www.allelefrequencies.net) was used to screen out a total of 55 HLA-A and HLA-B subtypes with a high proportion of Chinese population, which covered 98.47% of the Chinese population with HLA-A subtypes and 89.39% of that with HLA-B subtypes. The 55 HLA subtypes included 24 HLA-A subtypes and 31 HLA-B subtypes, specifically numbered as follows: HLA-A*01:01, HLA-A*02:01, HLA-A*02:02, HLA-A*02:03, HLA-A*02:04, HLA-A*02:05, HLA-A*02:06, HLA-A*02:07, HLA-A*03:01, HLA-A*11:01, HLA-A*11:02, HLA-A*23:01, HLA-A*24:02, HLA-A*24:03, HLA-A*26:01, HLA-A*29:02, HLA-A*30:01, HLA-A*31:01, HLA-A*32:01, HLA-A*33:01, HLA-A*33:03, HLA-A*66:01, HLA-A*68:01, HLA-A*68:02, HLA-B*07:02, HLA-B*08:01, HLA-B*13:01, HLA-B*13:02, HLA-B*15:01, HLA-B*15:02, HLA-B*15:10, HLA-B*27:02, HLA-B*27:03, HLA-B*27:05, HLA-B*27:06, HLA-B*35:01, HLA-B*38:01, HLA-B*38:02, HLA-B*39:01, HLA-B*39:09, HLA-B*39:011, HLA-B*40:01, HLA-B*40:02, HLA-B*40:06, HLA-B*44:02, HLA-B*44:03, HLA-B*46:01, HLA-B*48:01, HLA-B*51:01, HLA-B*52:01, HLA-B*53:01, HLA-B*54:01, HLA-B*55:02, HLA-B*57:01, and HLA-B*58:01.

A method for building the HLA-binding polypeptide database was as follows:

1. The HLA binding proteomes were immunoprecipitated, eluted and desalted

(1) 5×10⁷-10×10⁷ cells of each of the HLA subtype 721.221 cell lines were added to 2 ml of a protein lysis solution (including 20 mM of Tris [pH 8.0], 1 mM of EDTA, 100 mM of NaCl, 1% of Triton X-100, 60 mM of n-octylglucoside, PMSF, protease inhibitor, and 200 U of DNase), respectively; cell membranes were destroyed by sonicator (amplitude 35%, pulse 10 s) until no obvious precipitate was produced.

(2) The cell lysate was centrifuged for 20 min at 12,000 rpm and 4° C.; subsequently, the supernatant was extracted and co-incubated with 20 μl of GammaBind Plus Sepharose beads (bound to an anti-human HLA monoclonal antibody in advance) for 3 h.

(3) The beads were washed with a protein lysis solution without protease inhibitor and Triton X-100 four times, followed by washing with 10 mM Tris (pH 8.0) four times and distilled water once.

(4) The HLA-binding polypeptide proteomes were eluted and desalted with Empore C18 StageTips, where the StageTips was first washed with 100 μl of methanol twice, followed by washing with 50 μl of 50% acetonitrile/0.1% formic acid twice and 100 μl of 1% formic acid twice.

(5) The washed beads with the HLA-binding proteomes were dehydrated at 4° C., dried, and added with 50 μl of 3% acetonitrile/5% formic acid, and then added to the StageTips.

(6) The beads were washed with 50 μl of 0.1% formic acid again; the polypeptide proteomes were eluted from beads with 10% acetic acid, and the eluent was added to the StageTips.

(7) The StageTips was washed with 100 μl of 1% formic acid, and then, gradient eluents, i.e., 20 μl of 20% acetonitrile/0.1% formic acid, 20 μl of 40% acetonitrile/0.1% formic acid, and 20 μl of 60% acetonitrile/0.1% formic acid, were added, respectively.

(8) The above eluents were collected and dried.

2. HLA-binding proteomes obtained in the previous step were analyzed by high sensitivity protein mass spectrometry at an attomolar (10⁻¹⁸ molar) level, and the mass spectrometry data of the HLA-binding proteomes obtained from all of the 55 MHC-negative 721.221 cell lines was used to build the HLA-binding polypeptide database.

The specific implementations and embodiments of the present invention are described in detail above, but the present invention is not limited to the above implementations and embodiments. Within the knowledge of a person of ordinary skill in the art, various modifications can further be made without departing from the spirit of the present invention.

What is claimed is:

1. A tumor neoantigen prediction platform, comprising an HLA-binding polypeptide database.

2. The tumor neoantigen prediction platform according to claim 1, wherein the HLA-binding polypeptide database is built by analyzing binding proteomes of MHC-negative 721.221 cell lines expressing 55 HLA subtypes with a high proportion of Chinese population by a high sensitivity protein mass spectrometry at an attomolar level, and the 55 HLA subtypes comprise 24 HLA-A subtypes and 31 HLA-B subtypes, specifically numbered as follows: HLA-A*01:01, HLA-A*02:01, HLA-A*02:02, HLA-A*02:03, HLA-A*02:04, HLA-A*02:05, HLA-A*02:06, HLA-A*02:07, HLA-A*03:01, HLA-A*11:01, HLA-A*11:02, HLA-A*23:01, HLA-A*24:02, HLA-A*24:03, HLA-A*26:01, HLA-A*29:02, HLA-A*30:01, HLA-A*31:01, HLA-A*32:01, HLA-A*33:01, HLA-A*33:03, HLA-A*66:01, HLA-A*68:01, HLA-A*68:02, HLA-B*07:02, HLA-B*08:01, HLA-B*13:01, HLA-B*13:02, HLA-B*15:01, HLA-B*15:02, HLA-B*15:10, HLA-B*27:02, HLA-B*27:03, HLA-B*27:05, HLA-B*27:06, HLA-B*35:01, HLA-B*38:01, HLA-B*38:02, HLA-B*39:01, HLA-B*39:09, HLA-B*39:011, HLA-B*40:01, HLA-B*40:02, HLA-B*40:06, HLA-B*44:02, HLA-B*44:03, HLA-B*46:01, HLA-B*48:01, HLA-B*51:01, HLA-B*52:01, HLA-B*53:01, HLA-B*54:01, HLA-B*55:02, HLA-B*57:01, and HLA-B*58:01.

3. The tumor neoantigen prediction platform according to claim 2, wherein a method for building the HLA-binding polypeptide database comprises the following steps:
(1) performing an immunoprecipitation on the MHC-negative 721.221 cell lines expressing the 55 HLA subtypes with anti-human HLA monoclonal antibodies, and isolating the binding proteomes, followed by an elution and a desalting;
(2) analyzing the binding proteomes by the high sensitivity protein mass spectrometry at an attomolar level; and
(3) building the HLA-binding polypeptide database by mass spectrometry data of the binding proteomes.

4. The tumor neoantigen prediction platform according to claim 2, wherein each of the MHC-negative 721.221 cell lines expressing one of the 55 HLA subtypes is constructed by the following steps:
(1) replicating an HLA gene fragment from an HLA homozygous human B-lymphocyte cell line with HLA locus-specific primers, and validating the correctness of the sequence of an HLA PCR fragment;
(2) cloning the HLA PCR fragment into a retrovirus vector pLNCX2 and preparing a retrovirus; and
(3) infecting a 721.221 cell line with a cell culture medium containing the retrovirus, and picking out a resulted 721.221 cell line stably expressing an HLA subtype on a FACSAria cell sorter.

5. The tumor neoantigen prediction platform according to claim 1, wherein the tumor neoantigen prediction platform improves a tumor neoantigen prediction accuracy by optimizing a tumor neoantigen prediction algorithm, thus improving an efficiency of selection, research and development of vaccines.

6. The tumor neoantigen prediction platform according to claim 1, wherein the tumor neoantigen prediction platform is applied in a neoantigen vaccine development system.

7. The tumor neoantigen prediction platform according to claim 1, wherein the tumor neoantigen prediction platform is applied in oncology.

8. A method for constructing an MHC-negative 721.221 cell line expressing an HLA subtype, comprising the following steps:
(1) replicating an HLA gene fragment from an HLA homozygous human B-lymphocyte cell line with HLA locus-specific primers and validating the correctness of the sequence of an HLA PCR fragment;
(2) cloning the HLA PCR fragment into a retrovirus vector pLNCX2 and preparing a retrovirus; and
(3) infecting a 721.221 cell line with a retrovirus-containing cell culture medium, and picking out a resulted 721.221 cell line stably expressing the HLA subtype by a FACSAria cell sorter.

9. The method according to claim 8, wherein the method is applied in biomedicine.

10. The tumor neoantigen prediction platform according to claim 2, wherein the tumor neoantigen prediction platform improves a tumor neoantigen prediction accuracy by optimizing a tumor neoantigen prediction algorithm, thus improving an efficiency of selection, research and development of vaccines.

11. The tumor neoantigen prediction platform according to claim 3, wherein the tumor neoantigen prediction platform improves a tumor neoantigen prediction accuracy by optimizing a tumor neoantigen prediction algorithm, thus improving an efficiency of selection, research and development of vaccines.

12. The tumor neoantigen prediction platform according to claim 4, wherein the tumor neoantigen prediction platform improves a tumor neoantigen prediction accuracy by optimizing a tumor neoantigen prediction algorithm, thus improving an efficiency of selection, research and development of vaccines.


SEQUENCE LISTING (8 sequences)

SEQ ID NO: 1 - DNA, 31 bp, Artificial Sequence
  The sequence is synthetized.
tataaagcttgattctccccagacgccgagg

SEQ ID NO: 2 - DNA, 31 bp, Artificial Sequence
  The sequence is synthetized.
tataaagcttcacccggactcagagtctcct

SEQ ID NO: 3 - DNA, 31 bp, Artificial Sequence
  The sequence is synthetized.
atatgcggccgcacaaggcagctgtctcaca

SEQ ID NO: 4 - DNA, 31 bp, Artificial Sequence
  The sequence is synthetized.
atatgcggccgcatctcagtccctcacaaga

SEQ ID NO: 5 - DNA, 17 bp, Artificial Sequence
  The sequence is synthetized.
caggaaacagctatgac

SEQ ID NO: 6 - DNA, 20 bp, Artificial Sequence
  The sequence is synthetized.
taatacgactcactataggg

SEQ ID NO: 7 - DNA, 25 bp, Artificial Sequence
  The sequence is synthetized.
agctcgtttagtgaaccgtcagatc

SEQ ID NO: 8 - DNA, 26 bp, Artificial Sequence
  The sequence is synthetized.
acctacaggtggggtctttcattccc